SEQUENCE LISTING (6 sequences)

SEQ ID NO: 1 - protein, 430 aa, Mus musculus
MWRLVPLKLGRLSRALKLAALGSLLVLMLLHSPSLLASWQRNELADRRFLQLNKCPACFGTSWCRRFLNGQVGFETWGRLRLLDFLNVKNVYFAQYGEPREGGRRRVVLKRLGSQRELAQLDQSICKRATGRPRCDLLQAMPRTEFARLNGDVRLLTPEAVEGWSDLVHCPSQRLLDRLVRRYAETKDSGSFLLRNLKDSERMQLLLTLAFNPEPLVLQSFPSDEGWPFAKYLGACGRMVAVNYVGEELWSYFNAPWEKRVDLAWQLMEIAEQLTNNDFEFALYLLDVSFDNFAVGPRDGKVIIVDAENVLVADKRLIRQNKPENWDVWYESKFDDCDKEACLSFSKEILCARVTVDHNYYAVCQNLLSRHATWRGTSGGLLHDPPSEIAKDGRLEALLDECTNPKKRYGRFQAAKELRGYLAQLSHNVR

SEQ ID NO: 2 - protein, 430 aa, homo sapiens
MWRLVPPKLGRLSRSLKLAALGSLLVLMVLHSPSLLASWQRNELTDRRFLQLNKCPACFGTSWCRRFLNGQVVFEAWGRLRLLDFLNVKNVYFAQYGEPREGGRRRVVLKRLGSQRELAQLDQSICKRATGRPRCDLLQAMPRTEFARLNGDVRLLTPEAVEGWSDLVHCPSQRLLDRLVRRYAETKDSGSFLLRNLKDSERMQLLLTLAFNPEPLVLQSFPSDEGWPFAKYLGACGRMVAVNYVGEELWSYFNAPWEKRVDLAWQLMEIAEQLTNNDFEFALYLLDVSFDNFAVGPRDGKVIIVDAENVLVADKRLIRQNKPENWDVWYESKFDDCDKEACLSFSKEILCARATVDHNYYAVCQNLLSRHATWRGTSGGLLHDPPSEIAKDGRLEALLDECANPKKRYGRFQAAKELREYLAQLSNNVR

SEQ ID NO: 3 - DNA, 30 bp, Artificial sequence
  Chemically synthesized primer
ggccatttgcaaaatatcttggagcttgtg

SEQ ID NO: 4 - DNA, 29 bp, Artificial sequence
  Chemically synthesized primer
acttaactgtgccagatagccacgcagtt

SEQ ID NO: 5 - DNA, 30 bp, Artificial sequence
  Chemically synthesized primer
ggcggccatatggaccggcgcttcctgcag

SEQ ID NO: 6 - DNA, 43 bp, Artificial sequence
  Chemically synthesized primer
ggcggcggatccctacctcacgttgttacttaattgtgctagg

(19) United States
(12) Patent Application Publication
(10) Pub. No.: US 2012/0172306 A1
(43) Pub. Date: Jul. 5, 2012

(54) Use of HASF as a Protective Agent Against Ischemic Tissue Damage

(75) Inventors: Maria Mirotsou, Durham, NC (US); Victor J. Dzau, Durham, NC (US); Jing Huang, Vancouver (CA)

(73) Assignee: Duke University, North Carolina (US)

(21) Appl. No.: 13/319,487

(22) PCT Filed: Mar. 15, 2010
(86) PCT No.: PCT/US2010/027327

§ 371 Date: Mar. 15, 2012

Related U.S. Application Data
(60) Provisional application No. 61/159,845, filed on Mar. 13, 2009.

(51) Int. Cl. A61K 38/17; A61P 9/10

(52) U.S. Cl. 514/15.1

(57) ABSTRACT

A method of inducing a protective response against ischemic tissue damage is carried out by administering to subject a composition comprising an HASF polypeptide. The composition is administered prior to an ischemic event such as myocardial infarction to reduce tissue damage associated with such an event.

### RELATED APPLICATIONS

This application claims priority to U.S. Provisional Application No. 61/159,845, filed Mar. 13, 2009, which is incorporated herein by reference in its entirety.

### GOVERNMENT SUPPORT

This invention was made with Government support under NIH Grant R01-HL081744, R01-HL073219, R01-HL072010, and R01-HL035610. The Government has certain rights in the invention.

### FIELD OF THE INVENTION

The invention relates to tissue protection.

### BACKGROUND OF THE INVENTION

Myocardial infarction is a leading cause of death in America. As many as 50 million Americans have high blood pressure, the leading contributor to heart disease. Death due to cardiovascular disease is a worldwide problem with the highest death rates in the Soviet Union, Romania, Poland, Bulgaria, Hungary, and Czechoslovakia.

Stroke is currently the third leading cause of death in the US, behind heart disease and cancer. Each year, about 795,000 people in United States experience a new or recurrent stroke and in 2006 stroke accounted for approximately 1 of every 18 deaths in the United States

### SUMMARY OF THE INVENTION

The invention provides compositions and methods to reduce the level of tissue damage caused by an ischemic event such as a myocardial infarction or stroke, thereby reducing the death rate from such an event. A method of inducing a protective response against ischemic tissue damage is carried out by administering to subject a composition comprising an Hypoxia regulated Akt Mesenchymal Stem Cell (MSC) Factor (HASF), also known as “H12”, factor 12, or stem cell paracrine factor (SPF) prior to a prolonged/significant naturally-occurring or medically-induced ischemic event. HASF induces a physiological state that mimics ischemic preconditioning.

The subject to which HASF is administered is at risk of developing an ischemic event. The composition is administered to the subject prior to identification of major hypoxic event such as myocardial infarction or stroke. In some embodiments, the composition is administered before cell damage or identification of a symptom of ischemia or reperfusion injury. The compositions to be administered include HASF and a pharmaceutically acceptable excipient or carrier.

The subject is a risk candidate for an ischemic event or condition. For example, a subject is identified as having had a prior ischemic event such as a myocardial infarction, or is identified as having one or more risk factors such as family history of such events, smoking, high blood pressure, high blood cholesterol, diabetes, being overweight or obese, and physical inactivity. Symptoms of a cardiac event include for example, chest pain, arm pain, fatigue and shortness of breath. For example, the composition is administered at the onset of symptoms associated with a cardiac event such as a myocardial infarction or stroke. The composition is administered before or at the onset of or shortly after (e.g., within 3, 6, 12, 24 or 48 hours) of the onset of symptoms.

HASF used in the methods described herein is purified. A substantially pure HASF polypeptide (or fragment thereof) is preferably obtained by expression of a recombinant nucleic acid encoding the polypeptide or by chemically synthesizing the protein. A polypeptide or protein is substantially pure when it is separated from those contaminants which accompany it in its natural state (proteins and other naturally-occurring organic molecules). Typically, the polypeptide is substantially pure when it constitutes at least 60%, by weight, of the protein in the preparation. Preferably, the protein in the preparation is at least 75%, more preferably at least 90%, and most preferably at least 99%, by weight, HASF. Purity is measured by any appropriate method, e.g., column chromatography, polyacrylamide gel electrophoresis, or HPLC analysis. Accordingly, substantially pure polypeptides include recombinant polypeptides derived from a eucaryote but produced in E. coli or another procaryote, or in a eucaryote other than that from which the polypeptide was originally derived. Useful fragments of HASF are shorter than the full length mature protein and possess the cell protective effects of the full length protein. For example, the fragment is at least 10, 20, 50, 100, 200, or 300 amino acids in length and selectively induces PKCe expression of PKCe mediated cell survival.

The composition is administered systemically or locally. For example, the composition is administered directly, i.e., by myocardial injection to the cardiac tissue, or systemically, e.g., interperitoneally, orally, intravenously or by inhalation. In another example, administration of the composition is carried out by direct injection into the heart or by infusion into a coronary artery. Slow-release formulations, e.g., a dermal patch, in which diffusion of the composition from an excipient such as a polymeric carrier mediates drug delivery are also methods by which the composition is delivered.

For treatment of cerebral ischemia, HASF is optionally delivered locally to central nervous system (CNS) tissue, e.g., directly to brain tissue or infusion into cerebrospinal fluid. HASF is optionally delivered together with a blood-brain barrier permeabilization composition such as mannitol; mall fat-soluble molecules such as ethanol or ethanol derivatives; and water-soluble molecules such as glucose, mannitol, amino acids, dihydroxyphenylalanine, choline, and purine bases and nucleosides or derivatives thereof.

The compositions and methods are useful to induce a protective response against ischemic tissue damage, reducing ischemic damage to an organ, and/or reducing the level of apoptosis, by pre-emptive administration of a therapeutically effective amount of the composition. For example, HASF is administered at least one year prior to an ischemic event. For at risk patients, the patient is on a schedule of HASF for 1, 2, 3, 5, 10, 15, or 20 years prior to experiencing a significant or prolonged ischemic event. HASF is administered at least three times prior to an ischemic event. For example, the composition is administered daily, weekly, or monthly. Alternatively, HASF is administered immediately or shortly after occurrence of the ischemic event.

Standard routes of administration e.g., oral, intravenous, intranasal, subcutaneous, topical, intramuscular, and intraperitoneal delivery routes are used. HASF can also be administered directly to injured and damaged tissue (e.g., infarct and surrounding border zones). Such administration is particularly suitable to treat cardiovascular events, thus minimizing heart muscle injury or stimulating tissue repair processes in the heart after infarction. Other delivery systems and methods include, but are not limited to catheter-based devices that permit site specific drug delivery to the heart muscle, via a thorascopic opening (small minimally invasive wound in the thoracic cavity) through which a scope and guided injection device containing HASF is introduced, ultrasonic-based drug delivery methods, and infusion into the pericardial space.

Preferably, the tissue is cardiac or neuronal tissue. Alternatively, the tissue is a non-cardiac tissue such as kidney, brain, skeletal-muscle, lung, liver, or skeletal tissue. By treating a cardiovascular event or other ischemic condition, the disorder or condition is prevented or is delayed. Alternatively, tissue damage and its progression is slowed down, the extent of the injury is reduced, and the recovery is accelerated.

A therapeutically effective amount means the dose required to prevent or delay the onset, slow down the progression or ameliorate the symptoms of an ischemic disorder. Dosages depend on the disease state or condition being treated and other clinical factors, such as weight and condition of the subject, the subjects response to the therapy, the type of formulations and the route of administration. A suitable dose of a HASF for administration to adult humans ranges from about 0.001 mg to about 20 mg per kilogram of body weight. In some embodiments, a suitable dose is in the range of about 0.01 mg to about 5 mg per kilogram of body weight. Precise dosages to be therapeutically effective and non-detrimental are determined by those skilled in the art HASF is administered at a dose that increases the activity or tissue expression of phospho-ERK 1/2. In another embodiment, HASF is administered at a dose that increases the activity or tissue expression of protein kinase C epsilon (PKCe). The HASF composition comprises the amino acid sequence of SEQ ID NO:1 or 2. Alternatively, the HASF composition comprises a fragment of the full-length sequence (SEQ ID NO:1 or 2) that comprises the activity of increasing tissue expression of phospho-ERK or PKCε. The protein or peptide optionally contains conservation amino acid substitutions or other substitutions or alterations provided that the altered protein or peptide possesses the afore-mentioned tissue protective activities.

The compositions described herein are purified, e.g., synthetically produced, recombinantly produced, and/or biochemically purified. A purified composition such as a protein or peptide is at least 60%, by weight, free from proteins and naturally occurring organic molecules with which it is naturally associated. Preferably, the preparation is at least 75%, more preferably 90%, and most preferably at least 99%, by weight, the desired composition. A purified antibody may be obtained, for example, by affinity chromatography. By “substantially pure” is meant a nucleic acid, polypeptide, or other molecule that has been separated from the components that naturally accompany it. Typically, the polypeptide is substantially pure when it is at least 60%, 70%, 80%, 90%, 95%, or even 99%, by weight, free from the proteins and naturally-occurring organic molecules with which it is naturally associated. For example, a substantially pure polypeptide may be obtained by extraction from a natural source, by expression of a recombinant nucleic acid in a cell that does not normally express that protein, or by chemical synthesis.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, GenBank/NCBI accession numbers, patent applications, patents, and other references mentioned herein are incorporated by reference in their entirety. In the case of conflict, the present specification, including definitions, will control. In addition, the materials, methods, and examples are illustrative only and not intended to be limiting.

All references, e.g., faunal publications or the contents of Genbank accession numbers, are hereby incorporated by reference. Other features and advantages of the invention will be apparent from the following detailed description and claims.

### BRIEF DESCRIPTION OF THE DRAWINGS

FIG. 1A is a bar graph showing expression levels of HASF in mouse MSCs. Affymetrix microarray expression data of HASF in Akt-MSCs and control-MSCs under normoxia and/or 6 h hypoxia conditions. Y axis: Relative expression level in microarray gene chip. * indicates statistical significance of P<0.001. Values are Means±SD in triplicates.

FIG. 1B is a photograph of an electrophoretic gel. RT-PCR validation of mouse HASF expression in Akt-MSCs and control MSCs under normoxia and/or 6 h hypoxia conditions. A PCR fragment (626 bp) of mouse HASF was amplified by standard PCR. Mouse beta actin was used as the internal control. GFP: control GFP-MSCs, Akt: Akt-MSCs.

FIG. 1C is a photograph of an electrophoretic gel The full-length cDNA of human HASF without the stop codon was amplified by standard PCR and cloned in the Gateway Entry vector first for sequencing and then recombined in Destination vector 40 as V5 epitope tagged HASF at the carboxyl terminus. HEK 293 cells were transiently transfected with/without this expression construct. The supernatants from transfected cells were collected and probed with an anti-V5 antibody for western blotting. HASF protein was detected as ˜40 kDa protein bands in the supernatant of transfected HEK 293 cells, but not in the supernatant of control lipofectamine transfected cells. Lane 1, control lipofectamine transfected cells; lane 2 and 3, with HASF expression construct, 24 h and 48 h after transfection respectively.

FIG. 1D is a photograph of an immunoblot. Western blot for HASF expression and secretion levels in MSCs. Mouse MSCs were challenged with hypoxia for 4 hours. The cell lysates and conditional medium were collected and used to check the levels of HASP using HASF specific antibody. GFP: control GFP-MSCs, Akt: Akt-MSCs.

FIG. 2A is a bar graph showing % apoptosis. H9C2 cardiac myoblasts were pre-incubated ±10 nM of HASF recombinant protein for 30 min, and then challenged with 100 μM of H2O2 for 2 h. Apoptosis was quantified on a flow cytometer with Annexin V and Propidium Iodine (PI) staining. The sum of Annexin V positive cells+Annexin V/PI double positive cells is presented as percentage (%) of total cells. Human recombinant IGF protein was used as a positive control.

FIGS. 2B and 2C are bar graphs showing relative caspase activity. Adult rat cardiomyocytes were freshly isolated and treated ±10 nM of HASF for 30 min, and then challenged with 100 μM of H₂O₂ for various time points. H₂O₂ induced apoptosis in cardiomyocytes was evidenced by the dynamic increase in the activities of the initiator Caspase 9 and effector Caspase 317 at 5 h, 7 h and 9 h respectively. Y axis represent the relative amount of luminescence indicating the relative amount of active Caspase activities. ** P<0.01 and *** P<0.001. Data are presented as means±standard deviation of triplicates.

FIG. 2D is a series of photomicrographs and a bar graph showing that HASF protects from mPTP channel opening and cell death after ischemia and reperfusion. Adult rat cardiac myocytes were cold-loaded with fluorescent calcein and CoCl2 and subjected to 3-5 h of ischemia followed by 30-60 min reperfusion. Mitochrondria were counterstained with TMRM. Under these conditions increased calcein intensity (green) indicate the entrapped calcein in mitochrondria which is due to reduction of open mPTP channels. Upper panel: Representative images of cells subjected to 3 h ischemia, 60 min reperfusion and cells treated with 100 nM HASF prior to 3 h ischemia, 60 min reperfusion. Lower panel: Quantitative fluorescence analysis of images represented above. The percentage of Calcein intensity was normalized to mitotracker, mitochondrial, intensity and the value was inverted and presented as of mPTP channel opening. Data are presented as means±stdev of values estimated from 8 images per condition.

FIG. 2E is a bar graph showing caspase activity in primary neurons. Embryonic rat neuronal cells were pre-incubated ±100 nM of HASF recombinant protein for 30 min, and then challenged with 200 μM of H₂O₂ or 30 h. Apoptosis was determined by caspase 9 levels. The results from two independents experiments are shown. ** P<0.01. Data are presented as means±standard deviation of triplicates. These data indicate that HASF protects cardiomyocyte and neuronal cells against cell death.

FIG. 3A is a photograph of an electrophoretic gel. HASF activates the PKC pathway. A. Western blots analysis for various signaling proteins. Serum-starved adult rat cardiomyocytes were stimulated with 100 nM HASF recombinant protein for various time courses. Cell lysate was collected and the proteins were resolved by SDS-PAGE. Insulin from bovine pancreas was used as a positive control. min: minutes of treatment.

FIG. 3B is a photograph of an immunoblot. Serum-starved adult rat cardiomyocytes were pre-treated with 3 μM PKC inhibitors BIM, Gö6976, or Gö6983 for 30 minutes, then stimulated with 100 nM HASF recombinant protein for 5-10 min and cells were harvested. Total cell lysates were used to analyze the levels of ERK1/2 phosphorylation by western blot analysis.

FIG. 3C is a bar graph showing fold-change in caspase activity. Serum-starved adult rat cardiomyocytes were treated with 3 μM PKC inhibitors or vehicle DMSO for 30 minutes, then 100 nM HASF recombinant protein was added for 30 minutes followed by treatment with 200 μM H₂O₂ for 3 hours. Apoptosis was determined by detection of Caspase-9 activity using a homogeneous luminescent assay. All samples were measured in triplicates. *P<0.05 vs. non-HASF treated cells.

FIG. 4A is a photograph of an immunoblot. Western blot analysis for PKC E and PKC delta phosphorylation on total cell lysates from serum-starved adult rat cardiomyocytes stimulated with 100 nM HASF recombinant protein at 5 and 30 min. HASF b1 and b2 represent different batches of recombinant protein. PMA was used as positive control.

FIG. 4B is a photograph of an immunoblot. Western blot analysis for ERK1/2 phosphorylation on total cell lysates from adult rat cardiomyocytes pre-treated with 10 pM PKC epsilon translocation inhibitor peptide for 20 min, then 100 nM HASF recombinant protein was added for 5 min or 10 min.

FIG. 4C is a bar graph showing relative caspase activity in adult cardiomyocytes. Apoptosis as determined by detection of Caspase-9 activity on adult rat cardiomyocytes pre-treated with 178 μM PKC translocation inhibitor peptide for 10 minutes and then with 100 nM of HASF for 30 minutes. Thereafter, cells were challenged with 200 μM H₂O₂ for 3 hours. All samples were measured in triplicates. *P <0.05 vs. non-HASF treated cells and the experiment was repeated multiple times.

FIG. 4D is a bar graph and a photomicrograph showing the effect of recombinant protein, HASF on mPTP channel as imaged by confocal microscopy. Images demonstrate adult rat cardiomyocytes pre-treated with or without PKC epsilon inhibitor peptide for 10 min and with HASF for 30 min in normoxia followed by hypoxia (4 hours) and re-oxygenation (30 min) at 37 C. The presence of PKCepsilon inhibitor peptide caused the opening of the mPTP channel as observed by decreased (quenched) calcein intensity. The bar graph depicts measured fluorescence of calcein versus mitotracker intensity under different conditions. Mitrotracker (red); mitochondrial marker, unquenched calcein (green) in mitochondria. A high magnification (63×) of a representative cardiomyocyte treated with recombinant protein, HASF subjected to hypoxia/re-oxygenation is shown.

FIG. 4E is a bar graph showing relative caspase activity in primary neurons. Apoptosis as determined by detection of Caspase-9 activity on primary neuron pre-treated with 178 μM PKC translocation inhibitor peptide for 10 minutes and then with 100 nM of HASF for 30 minutes. Thereafter, cells were challenged with 200 μM H₂O₂ for 3 hours. All samples were measured in replicas N-4 and the experiments was repeated twice. *P<0.05 vs. non-HASF treated cells. These data show that PKC epsilon plays an essential role in HASF-mediated ERK activation and anti-apoptotic effects.

FIG. 5A is a series of photographs and a bar graph showing infarct area. Rats were randomly divided into PBS control group and HASF injected group, n=10 for each group. The reperfusion injury model was achieved by 30 min coronary ligation with the immediate injection of vehicle PBScontrol or 1 μg of SPF recombinant protein into the myocardium area below ligation suture, followed by loosening of the ligation suture to achieve reperfusion injury. Area at risk (AAR) was calculated as the left ventricular total area that did not stain Evans Blue dye, and % infarct area was calculated as the % of infarct area/AAR. The mean of % of infarct area for all sections of each heart was calculated blindly for comparisons using ImageJ computer software. Representative photos from each group are shown.

FIG. 5B is a series of photographs and a bar graph showing TUNEL staining in control or HASF injected animals from groups described above to detect in vivo apoptosis of cardiomyocytes. Serial cyrosections of 5 μm thick were made immediately below the ligation area, 10 sections for each heart were analyzed, with N=8 rats per group. Sections were counterstained with hematoxylin. Total number of dark-brown color stained apoptotic nuclei were counted and added up blindly in 10 randomly taken fields within the peri-infarct region in each group.

FIG. 5C is a series of photographs and a bar graph showing fibrosis. For fibrosis analysis, animals were sacrificed 4 weeks after the initial ischemia/reperfusion injury and serial cryosections of 5 μm thick were made immediately below the ligation area, 10 sections for each heart were analyzed. HASF injected group N=8 rats. PBS injected control group N=6. Brilliant blue color stained collagen area was quantified using ImageJ computer software and the mean of % fibrosis was calculated as collagen positive area/total area. *** P<0.001. These data indicate that HASF decreases cardiomyocyte apoptosis and reduces infarct size and fibrosis in vivo.

FIGS. 6A and 6B are photographs of electrophoretic gels showing HASF expression in bacterial cells. FIG. 6A shows Comassie staining of the expression of HASF recombinant protein. The open reading frame of human HASF without N-signal region (1158 bp) was re-cloned into pET 15b vector to generate a 6×His-HASF recombinant protein, This novel HASF protein was cysteine-rich and expressed exclusively as a ˜40 KDa protein in the ‘inclusion bodies’ 3 h after induction of 1 mM of IPTG at 28° C. Lane 1, protein marker; lane 2, before induction; lane 3, 3 h after induction; lane 4, insoluble fraction as inclusion bodies; lane 5, soluble fraction. FIG. 6B shows Comassie staining of recombinant 6×His tagged HASF protein after purification and refolding. Lane 1, protein marker; lane 2-7, increasing amount of 6×His tagged HASF recombinant protein from 50 ng up to 500 ng after refolding.

FIGS. 7A-C are a series of photographs showing the effects of HASF in cytochrome c, Bcl2, Bax and DNA degradation. In FIG. 7A, Adult rat cardiomyocytes were treated with +10 nM of HASF for 30 min, and then challenged with 100 μM of H₂O₂ for 6 h. Mitochondrial fraction or cytosolic fraction or total cell lysate were extracted and separated in 15% SDS-PAGE and transferred to nitrocellulose membrane and probed with mouse anti-Cytochrome C monoclonal antibody. Lane 1-2, —H₂O₂ control; lane 3-4, +H₂O₂ control; lane 5-8, four individual samples of +HASF recombinant protein and then +H₂O₂. In FIG. 7B, Western blot of mitochondrial fractions with Bcl2 and Bax antibodies respectively. Lane 1, —H₂O₂control; lane 2, +H₂O₂control; lane 3-6, four individual samples of +HASF and then +H₂O₂. In FIG. 7C, adult rat cardiomyocytes were treated +10 nM of HASF for 30 min, and then challenged with 100 μM of H₂O₂ for overnight (˜15 h). Genomic DNA was extracted and separated on 1% agarose gel. H₂O₂ induced apoptosis in cardiomyocytes was companied by typical DNA fragmentation (laddering) at late-stage apoptosis. Pre-incubation of cardiomyocytes with 10 nM of this HASF recombinant protein inhibited the DNA laddering to a noticeable extends. Lane 1, DNA marker; lane 2, —H₂O₂control; lane 3-4, +H₂O₂; lane 5-6, +HASF recombinant protein and then +H₂O₂.

FIGS. 8A-B are photographs of immunoblots. Western blot analysis for PKC a/b and PKC theta phosphorylation on total cell lysates from serum-starved adult rat cardiomyocytes stimulated with 100 nM HASF recombinant protein for the indicated times. HASF batch 1 and 2 represent different batches of recombinant protein. PMA was used as positive control.

FIG. 9 is a table showing PKC inhibitors.

FIG. 10 is a comparison of the amino acid sequence of human and mouse HASF.

FIG. 11 is a diagram showing mechanism of action of HASF protection.

### DETAILED DESCRIPTION

Ischemic preconditioning is a process by which the extent of damage to the myocardium is decreased following a prolonged or significant ischemic event such as myocardial infarction. The process of ischemic preconditioning reduces damage to the heart by subjecting the heart to short bouts of ischemia prior to a prolonged or significant ischemic episode. Preconditioning reduces damage from unpredictable or naturally-occurring ischemic events such as acute myocardial infarction, stable angina, cardiac stunning, and myocardial hibernation. The process is also useful to reduce damage to the myocardium when the time of cardiac ischemia is predictable, such as the damage occurring during bypass surgery, cardiac transplantation, and elective angioplasty.

Since subjecting patients to short bouts of ischemia is often not a viable clinical approach, the compositions and methods of the invention induce one or more of the physiological elements of the preconditioning phenomenon to confer a clinical benefit in a safe, unstressful manner.

PKC epsilon is involved in preconditioning-mediated cardioprotection and is a critical mediator of this process. HASF has been found to induce the beneficial events of the pre-conditioning process. HASF mediated cardiac protection is modulated via PKCε in a high specific manner. HASF activated PKCε and is highly specific to this isoform of the PKC protein family. Prior to the event, few or no specific activators of the PKCε were known. This specificity is important, because PKCε play a key role in protective preconditioning (a physiological phenomenon with high clinical implications for cardiac disease), whereas an increase in activity or expression of some other PKC isoforms (e.g., PKC δ) in undesirable due to its association with increased tissue damage.

Also useful is the autocrine effect of HASF in MSCs. MSCs that overexpress HASF are protected from ischemic conditions (thus improving the efficiency of such stem cell therapy). Such MSCs survive better in the ischemic myocardium through autocrine mechanisms that precondition those cells.

### Signs and Symptoms of an Ischemic Event

Signs and symptoms of myocardial infarction or heart attack include chest discomfort, or discomfort elsewhere in the upper body as well as shortness of breath with or without chest discomfort. Most heart attacks involve discomfort in the center of the chest that lasts more than a few minutes, or that goes away and comes back and/or uncomfortable pressure, squeezing, fullness or pain. Discomfort in other areas of the upper body can include pain or discomfort in one or both arms, the back, neck, jaw or stomach. Other signs may include breaking out in a cold sweat, nausea or lightheadedness. Clinical diagnosis of such an event is well known in the art, including, e.g., electrocardiogram (ECG) testing and cardiac enzyme (e.g., creatine kinase, troponin T, and troponin I) testing.

Signs and symptoms of a stroke or transient ischemic attack (TIA), include: sudden numbness or weakness of the face, arm or leg, especially on one side of the body; sudden confusion, trouble speaking or understanding; sudden trouble seeing in one or both eyes; sudden trouble walking, dizziness, loss of balance or coordination; and/or sudden, severe headache with no known cause. Clinical diagnosis of stroke is also well known in the art, e.g., computed tomography (CT) scan and/or magnetic resolution imaging (MRI) scan.

HASF is administered before or shortly after such ischemic events.

### HASF Protects Ischemic Cardiacmyocytes Via the PCK-ERK Pathway Mediated Mechanisms

MSCs overexpressing Akt release several paracrine factors to promote cardiac repair after acute myocardial infarction (AMI). HASF, a.k.a. SPF, factor 12 or H12, was identified as dramatically upregulated in Akt-MSCs in response to hypoxia. Addition of recombinant HASF protected cardiomyocytes against oxidative stress induced apoptosis in vitro and markedly reduced the size of MI in a heart ischemia/reperfusion animal model.

HASF protects cardiomyocytes and neuronal cells against stress induced apoptosis and inhibits mitochondrial PTP opening after hypoxia-reoxygenation by specific activation of PKC epsilon. Indeed, selective inhibition of PKCe blocks HASF induces cell protection. In vivo administration of HASF reduces ischemia-reperfusion myocardial damage. HASF is the first protein identified that selectively activates PKCe, a member of the PKC family specifically implicated in preconditioning associated cytoprotection. The data described herein indicate that HASF plays important physiological and therapeutic roles in stem cell mediated tissue protection, repair and regeneration.

To address the mechanism underlying the protective effects of HASF treatment, several cell signaling pathways that play pivotal role in determination of cell fate during apoptosis were examined. HASF was found to activate the PKC-ERK pathway. These effects were exerted via specific activation of the PKC epsilon isoenzyme, an isoform of the novel PKC subfamily, associated with protective signaling processes during in ischemic preconditioning. Thus, HASF is useful as a therapeutic strategy in patients with myocardial ischemia/reperfusion injury and other injuries associated with ischemic cell death.

### HASF Selectively Activated PKC Epsilon Mediated Cell Survival and Prevents Ischemic Death

HASF protects cardiomyocytes and neuronal cells against stress induced apoptosis and inhibits mitochondrial PTP opening after hypoxia-reoxygenation by specific activation of PKCε. Indeed, selective inhibition of PKCε blocks HASF induces cell protection in vitro. In vivo administration of HASF reduces ischemia-reperfusion myocardial damage. HASF is the first protein that selectively activates PKCε, a member of the PKC family specifically implicated in preconditioning associated cytoprotection. The data described herein that HASP plays important physiological and therapeutic roles in stem cell mediated tissue protection, repair and regeneration.

The following materials and methods were used to generate the data described herein.

### Bioinformatics and Molecular Biology

GeneChip Mouse Genome 430A 2.0 Array (Affymetrix, Inc.) was used to discover differentially expressed novel transcripts in mouse Akt-MSCs. Novel transcripts and the predicted protein sequences from Akt-MSCs were assessed for being secreted proteins by the prediction of possessing a N-signal peptide and the exclusion of transmembrane domains. A PCR fragment (626 bp) of mouse HASF was amplified from mouse Akt-MSCs with the forward primer, 5′-ggccatttgcaaaatatcttggagcttgtg-3′ (SEQ ID NO:3) and reverse primer, 5′-acttaactgtgccagatagccacgcagtt-3′ (SEQ ID NO:4). This PCR product was subsequently cloned into pGEM-TA vector (Promega) for sequencing and was on the other hand, labeled with ³²P isotope as the probe for northern blotting (Ambion, FirstChoice Mouse blot 1). Human homologous cDNA of HASF, with gene name as chromosome 3 open reading frame 58, (C3orf58) was purchased from American Type Culture Collection (ATCC, clone MGC 33365 or IMAGE 5267770). Full-length human cDNA of HASF without the stop condon was amplified by PCR and cloned in Gateway Entry vector for sequencing and subsequently recombined into Gateway destination vector 40 (Invitrogen) as the mammalian expression construct to generate the V5-epitope tagged HASF for transfection and detection in the culture medium of HEK293 cells by western blotting with rabbit anti V5 antibody (Abeam).

### Recombinant Protein Purification, Refolding and Mass Spectrometry

The open reading frame of human HASF without the predicted N-signal sequence (1158 bp) was cloned in-frame in pMal-2C vector. The same open reading frame of human HASF (1158 bp) without N-signal sequence was next amplified with the forward primer (underlined with Nde I restriction site), 5′-ggcggccatatggaccggcgcttcctgeag-3′ (SEQ ID NO:5) and the reverse primer (underlined with BamH I restriction site), 5′-ggcggcggatccctacctcacgttgttacttaattgtgctagg-3′ (SEQ ID NO:6), which was cloned in-frame into pET 15b vector (EMD Biosciences) to generate 6×His tagged HASF recombinant proteins. The expression of this 6×His-HASF recombinant protein was induced for 3 h at 28° C. by adding 1 mM of IPTG in E. coli. BL21 (DE3) strain when the OD600 reached 0.6 and expressed exclusively in inclusion bodies, which after washing and re-centrifuging extensively in large volume of 20 mM of Tris (pH 7.5), 10 mM of EDTA and 1% Triton X-100 for 6 times, protein pellet was subsequently solublized in a denaturing buffer containing 50 mM CAPS (pH11.0) and 0.2% of N-lauroylsarcosin, and refolded by extensive dialysis in 20 mM of Tris (pH 8.0) and 20 mM of NaCl with step-wise decreasing amount of dithiothreitol starting at 200 μM at 4° C. Promotion of intramoleculer disulfide bonds was further enhanced by adding a redox pair of 0.2 mM of oxidized v.s.1 mM of reduced glutathione at room temperature. Misfolded recombinant proteins were then precipitated and removed by centrifugation for 30 min at 4° C. Soluble recombinant proteins were further enriched through TALON affinity chromatography (Clontech) and after elution with 1 M of imidazole, pH 7.0, this 6×His-HASF recombinant protein was finally dialyzed at 4° C. overnight in a large volume of phosphate buffered saline (PBS), pH 7.4 and concentrated by centrifugation through the filtration tubes with 3 KDa molecular weight cut-off membranes (Sartorious/Vivascience) at 4° C. The 6×His HASF recombinant proteins were then immediately stored at −80° C. in small aliquots and thawed only once for experiments. To confirm the protein sequences, the 6×His-HASF recombinant protein were subsequently digested with trypsin (0.6 μg), and the tryptic peptides were subjected to matrix-assisted laser desorption-ionization mass spectrometry (MALDI-MS) on an Applied Biosystems 4700 Proteomic Analyzer® time of flight (TOFTOF®) mass spectrometer. Positive mode time of flight was used to identify peptides, and individual peptides were sequenced by MS/MS using collision-induced dissociation. All sequence and peptide fingerprint data was searched using the SwissProt database and Mascot search engine.

### In Vitro Annex V/PI Staining

Rat cardiac myoblasts-H9C2 cells were obtained from ATCC and cultured in DMEM medium containing 10% of FBS, supplemented with 2 mM of L-glutamine, 100 U/ml of penicillin and 100 μg/ml of streptomycin (Invitrogen). Cells were seeded one day before at 1×10⁵/well in 6-well plates. The following day, the cells were treated for 30 min with 10 nM of HASF the recombinant protein, the MBP or PBS were used as controls. Then the cells were challenged with 100 μM of H2O2 for 2 h. The attached and floating cells were collected. H2O2 induced apoptosis was then analyzed on a flow cytometer for Annexin V/Propidium Iodine double staining with the Vybrant Apoptosis Assay Kit #2 (Invitrogen).

### Caspase Assays, DNA Fragmentation and Apoptosis-Related Genes Expression by Western Blotting

Adult rat ventricular cardiomyocytes were isolated from 6 weeks old female Sprague-Dawley rat (Harlan World Headquarters, Indianapolis, Ill., USA) hearts by enzymatic digestion and were seeded in (6-well) plates or Delta T culture dishes (Bioptechs, Inc., Pa.) pre-coated with 1 μg/cm2 of laminin (Sigma) at 5×10⁴/well and cultured overnight in serum-free M199 medium (Sigma), supplemented with 2 mM of L-carnitine, 5 mM of creatine, 5 mM of taurine, 0.2% of albumin, 100 U/ml of penicillin and 100 μg/ml of streptomycin. Cells were briefly treated without or with PKC inhibitor (with vehicle saponin), then recombinant protein HASF (10 or 100 nM) was added into cells for 30 min, with PBS used as vehicle controls. The cells were challenged with H₂O₂ for various time points. For the Caspase assays, cardiomyocytes were scraped off plates in lysis buffer and were analyzed by a luminescent plate reader with Caspase-Glo 3/7 and or Caspase-Glo 9 kits (Promega); and for DNA fragmentation, genomic DNA from cardiomyocytes was extracted and separated on 1% agarose gel electrophoreses, with Apoptotic DNA Ladder Extraction Kit (BioVision), according to manufacturers' instructions. For western blotting of apoptosis-related gene expression, mitochondrial and cytosolic fraction of cell lysate were extracted, the proteins were then separated on a 15% SDS-PAGE and transferred to nitrocellulose membrane (Biorad), probed with mouse anti-Cytochrome C monoclonal antibody (Calbiochem), rabbit anti-Bcl-2 polyclonal antibody (Abeam), or rabbit anti-Bax polyclonal antibody (Abeam), and with rabbit anti-mouse or goat anti-rabbit secondary antibodies (Abcam), respectively. For the experiments with the PKC epsilon inhibitor, cells were treated with 178 μM in 5 μg/ml of saponin for 10 min at 37° C. The inhibitor was removed and the cells were further treated with recombinant protein HASF (100 nM) for 30 min at 37 C and then subjected to H₂0₂ treatment. Then cells were lysed with lysis buffer and the collected lysate was used to measure caspase 9.

### Neuron Cells

Embryonic rat cortex tissue was purchased from Neuromics (Edina, Minn.). Primary neurons were isolated, cultured on poly-D-lysine coated glass slides according to provided instructions and allowed to grow for 5 days in complete neurobasal media. Cells were treated with PKC-epsilon inhibitor and recombinant protein HASF as described above for cardiomyocytes). For induction of stress the cells were challenged with H₂0₂ (50 μM) for 30 min followed by 3 hours of recovery at 37° C. Then cells were lysed with lysis buffer and the collected lysate was used to measure caspase 9 as described above.

### Mitochondrial Permeability Transition Pore (MPTP) Channel Opening

Adult rat cardiomyocytes were isolated and plated on culture dishes as previously described. The following day the cells were washed briefly with the Krebs-RingerHEPES (KRH) buffer (in mM, 115 NaCl, 5 KCl, 1 CaCl2, 1 KH2PO4, 1.2 MgSO4 and 25 HEPES buffer (pH 6.2)) and treated with or without PKC epsilon translocation inhibitor peptide with vehicle (saponin 5 ug/ml) for 10 min at 37′C. The inhibitor was removed and the cells were further treated with recombinant protein HASF (100 nM) for 30 min at 37 C. The cells were transferred to an anaerobic, hypoxia, chamber (Coy Laboratory Products, Ann Arbor, Mich.) for 4 hours as maintained under an atmosphere of 0.5% O2 and 95% N2. The fluorescent Calcein, CoCl2, mitotracker and Hoechst (Molecular Probe) dyes were added as the cells were in hypoxia chamber. The cells were re-oxygenated in full cardiomyocyte culture media (see above) at 37° C. for 30 min and images were captured by Zeiss LSM 510 confocal microscope. Alternatively, Adult rat cardiomyocytes were isolated as previously described and plated on culture plate. The following day the cells were washed with Hank's balanced salt and the cells were treated with or without PKC epsilon inhibitor translocation peptide (10 uM) with vehicle Saponin (5 ug/ml) for 30 min. Thereafter, the HASF protein (100 nM) was added into the cell media for additional 30 min at 37° C. prior to transferring the cells to an anaerobic, hypoxia, chamber (Coy Laboratory Products, Ann Arbor, Mich.) for 6 hours as maintained under an atmosphere of 0.5% O₂ and 95% N2. Fluorescent Calcein, CoCl2 and mitotracker were added to cells in hypoxia chamber. The cells were removed for re-oxygenation at 37° C. for different time courses and images were taken by Zeiss fluorescent microscope.

In Vivo model of Ischemia/Reperfusion Injury, Infarct Size, TUNEL and Fibrosis Assays

Female Sprague-Dawley rats were used for all in vivo experiments. A midsternal thoracotomy was performed to expose the anterior surface of the heart after anesthesia. The proximal left ascending coronary artery (LAD) was identified and a 6.0 suture (Ethicon) was placed around the artery and surrounding myocardium. Regional left ventricular ischemia was induced for 30 minutes by ligation of LAD, followed by immediate injection of 1 μg of recombinant protein HASF or PBS vehicle control in five spots of intramyocardium in a total volume of 250 μl. The ligature was loosened and reperfusion was achieved after 30 min of the ischemia period and the incision was closed and the animals were allowed to recover.

For analysis of infarct size, 24 h after reperfusion, the LAD was re-ligated and ˜300 μl of 1% Evans Blue in PBS (pH 7.4) was retrogradely infused into the heart in a 2-3 min period to delineate the non-ischemic area. The heart was excised and rinsed in ice-cold PBS. Five biventricular sections of similar thickness were made perpendicular to the long axis of the heart and incubated in 1% triphenyl tetrazolium chloride (TTC, Sigma) in PBS (pH 7.4) for 15 minutes at 37° C. and photographed on both sides. Area at risk (AAR) was calculated as the left ventricular total area excluding Evans Blue dye positive area, and % infarct area was calculated as the % of infarct area/AAR. The mean of % of infarct area for all sections of each heart was calculated blindly for comparisons using ImageJ computer software, with 10 rats in each group.

For TUNEL staining (DeadEnd Colometric TUNEL System, Promega) after 30 min ischemia/24 h reperfusion, serial cyrosections of 5 μm thick were made immediately below the ligation area, 10 sections for each heart were analyzed, with 8 rats in each group. Briefly, cryosections were first fixed in cold methanol for 5 min, washed in PBS and treated with proteinase K for 30 min at room temperature. Biotinylated nucleotide mix and rTdT enzyme were added to catalyze the end-labeling reaction for 1 h at 37° C. Streptavidin-HRP and DAB chromogen components were added to allow colormetric development. Sections were also counterstained with hematoxylin. Negative control was carried out with the same procedure except for adding rTdT enzyme. Total number of dark-brown color stained apoptotic nuclei were counted and added up blindly in 10 randomly taken fields within the peri-infarct region in each group.

For fibrosis analysis, animals were sacrificed 4 weeks after the initial 30 min ischemia/24 h reperfusion injury and serial cyrosections of 5 μm thick were made immediately below the ligation area, 10 sections for each heart analyzed, and with 8 rats in HASF protein injected group and 6 rats in PBS injected control group. Collagen deposition within the infracted region was stained with Masson's Accustain Trichrome Stains (Sigma) according to manufacturer's instructions. Brilliant blue color stained collagen area was quantified using ImageJ computer software and the mean of % fibrosis was calculated as collagen positive area/total area.

### Antibodies.

HASF-specific antibodies were made as follows. 6×His tagged human HASF recombinant protein from bacteria was generated, purified, and injected into a rabbit subcutaneously to raise antibody. Injections were done every 4-6 weeks, with bleeds 7-10 days after each injection. Immunized sera were collected, and IgG were purified with the Melon Gel IgG Spin Purification Kit (Pierce). The antibody for HASF was further characterized by immunoreaction with purified HASF protein and HASF gene transfected HEK293 cells, and HASF protein activity neutralization. The proper dilution was also determined.

Rabbit polyclonal antibodies for phospho-pan PKC, phospho-ERK1/2, phospho-raf, phosphor-PDK1, total Akt were obtained from Cell Signaling Tech. Rabbit polyclonal antibody for phospho-PKC epsilon, phospho-Bad, and GAPDH was obtained from Abeam. HRP-conjugated goat anti-rabbit secondary antibody was obtained from Cell Signaling Tech.

### PKC Inhibitors.

Bisindolylmalemide Hydrochloride (BIM/GF109203X) was ordered from Sigma. Gö6976, Gö6983, PKC ε translocation inhibitor peptide and its negative scramble control peptide were ordered from Calbiochem.

### Statistics

All the results are presented as the mean±SD or mean±SEM and were analyzed using unpaired student t test.

### HASF Increases Survival of Cardiac Myocytes and Neuronal Cells

Purified recombinant HASF protein was made from both bacterial and mammalian cell protein expression systems and the effects on cardiac myocytes and primary neuronal cells was evaluated. The data show that HASF increases the survival of cardiac myocytes and neuronal cells from stress and/or ischemia-reperfusion induced apoptosis and that this cytoprotective action is mediated through the PKC/ERK pathway. Specifically, PKCe, an isoform of the novel PKC subfamily, known to play a crucial role in the protective signaling processes during ischemic preconditioning, was upregulated by HASF treatment. Selective inhibitors of PKCe abated the HASF mediated cellular survival effects. In vivo administration of HASF reduced myocardial damage and enhanced repair after ischemic injury. These effects on cardiac myocytes and neuronal cells indicates that HASF plays a broad physiologic paracrine role in cell survival and is useful for therapeutic intervention in the treatment of ischemia reperfusion tissue injury and prevention of tissue damage associated with such injury.

### Characterization of HASF, a MSC Secreted Protein

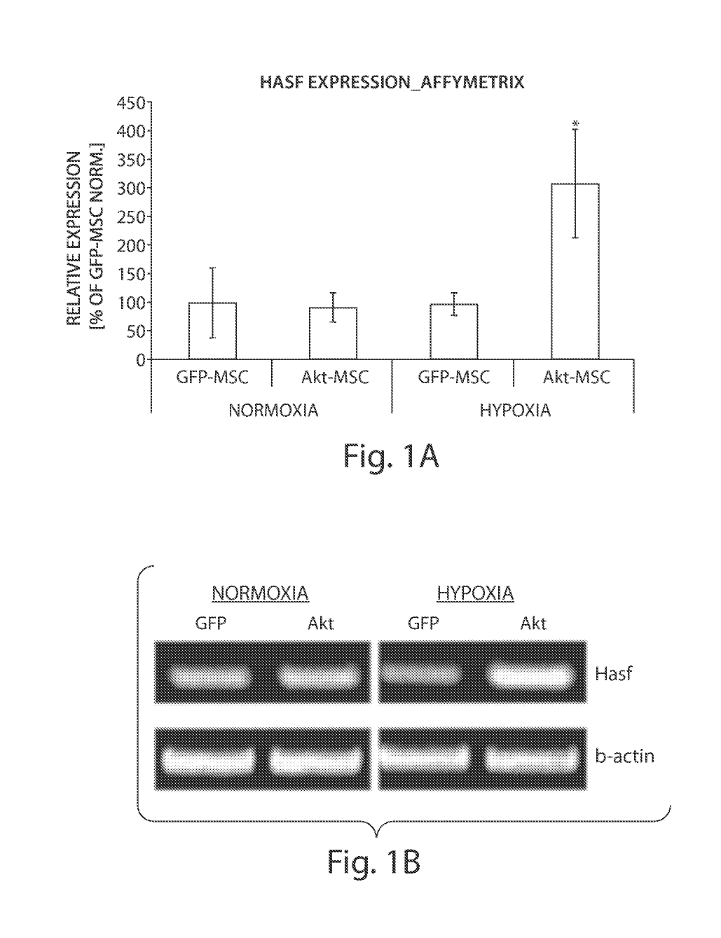

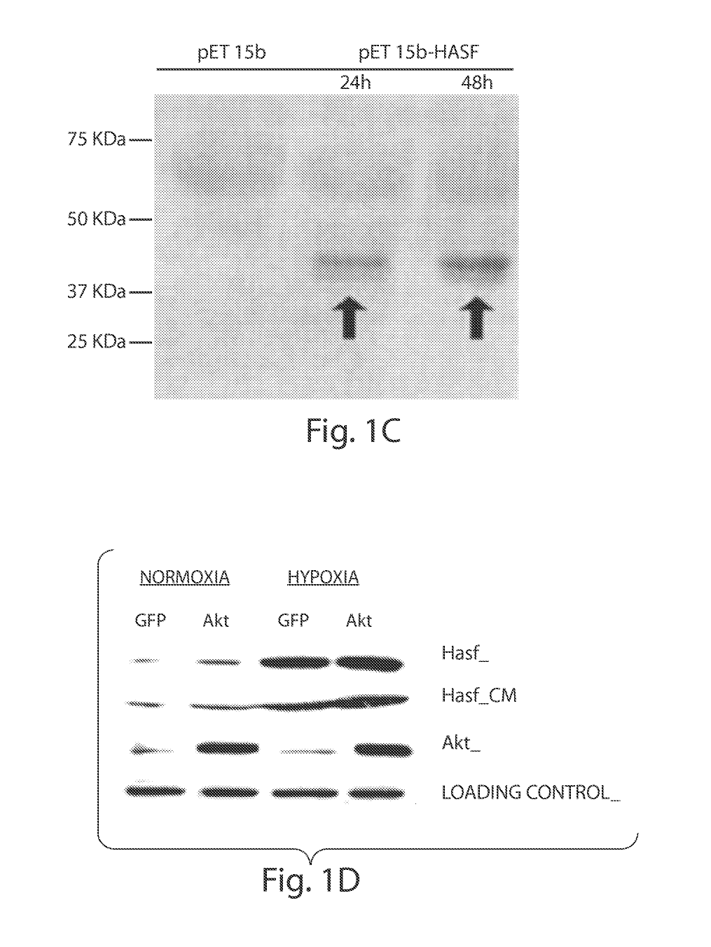

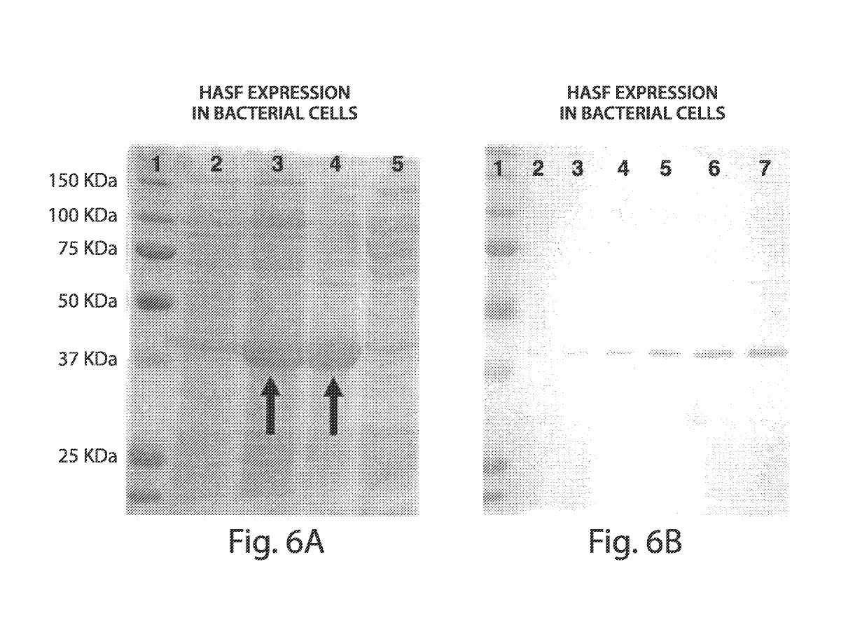

HASF), was dramatically upregulated in Akt-MSCs under hypoxic conditions (FIG. 1A). Bioinformatic analysis indicated that HASF is likely to be a secreted protein, with the first <45 amino acids as the N-signal peptide, without any O-/N-glycosylated sites and transmembrane domains predicted. Gene ontology prediction of human HASF (Genebank accession no. 205428) revealed that it is identical to gene C3orf58, chromosome 3 open reading frame 58, which has been associated with autism. The alignment with both human HASF and mouse HASF protein sequences (Genebank accession no. 68861, with gene name as 1190002N15Rik) revealed a highly conserved homology of about 98%. To further characterize HASF, a PCR fragment of 626 bp of mouse HASF was amplified from mouse Akt-MSCs under hypoxia and cloned into pGEM TA vector for sequencing. The sequences were exactly identical to the corresponding nucleotide positions 885-1484 of the mouse gene. Moreover, the open reading frame of human HASF without the N-signal region (1158 bp) was cloned into a pET 15b vector to generate a 6×His-HASF bacterial recombinant protein. As expected the protein was expressed exclusively in the inclusion bodies of E. coli. This 6xHis-HASF recombinant protein was then solublized first in denaturing condition and refolded with step-wise decreasing amount of dithiothreitol and a redox pair to promote disulfide bond formation (FIGS. 6A-C). Using this approach, 100 μg of high purity 6×His-HASF recombinant protein was produced from 500 ml of induced bacterial culture (yield 0.2 μg/ml). The protein sequence of this 6xHis-HASF recombinant protein was further confirmed by mass spectrometry.

To verify that HASF is a secreted protein, the full length cDNA of human HASF excluding the stop codon ‘TAG’ (1290 bp) was cloned to Gateway system and the vector was transfected in HEK cells to produce carboxyl-end-V5-polyhistidine tag 6×His epitope tagged HASF. Western blotting with rabbit anti V5 antibody confirmed the presence of V5-epitope tagged HASF in the culture media of HEK293 cells at 24 h and 48 h after transfection, but not in the medium of the vehicle control lipofectamine transfected HEK293 cells (FIG. 1B), indicating that HASF is a secreted protein.

Mouse HASF expression in Akt-MSCs and control MSCs under normoxia/hypoxia was verified using reverse transcript PCR(RT-PCR) amplification of the above PCR fragment (FIG. 1C). HASF mRNA expression pattern was consistent with the result of Affymetrix microarray expression data, demonstrating that mouse HASF is dramatically up-regulated in Akt-MSCs under hypoxic condition. In addition, western blot analysis, using rabbit polyclonal anti-HASF antibody, confirmed that HASF protein expression and secretion was increased in mouse Akt-MSCs under hypoxia condition (FIG. 1D).

### HASF Protects Cardiomyocyte and Primary Neuronal Cells Against Cell Death

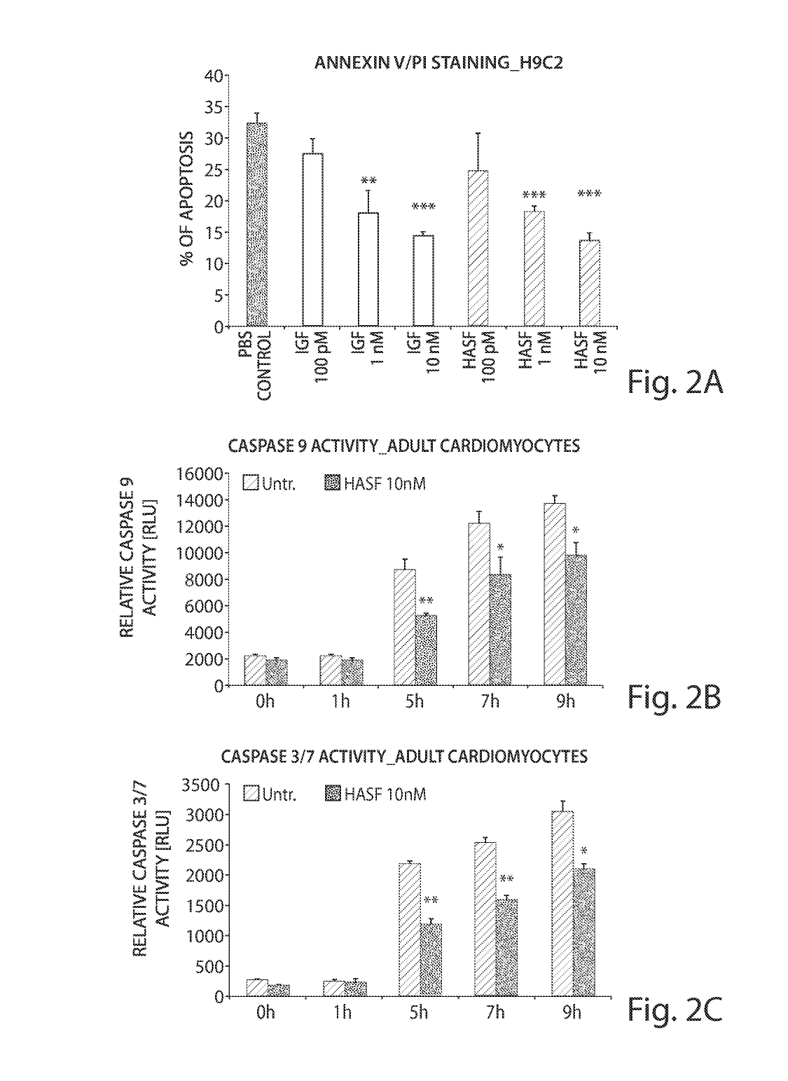

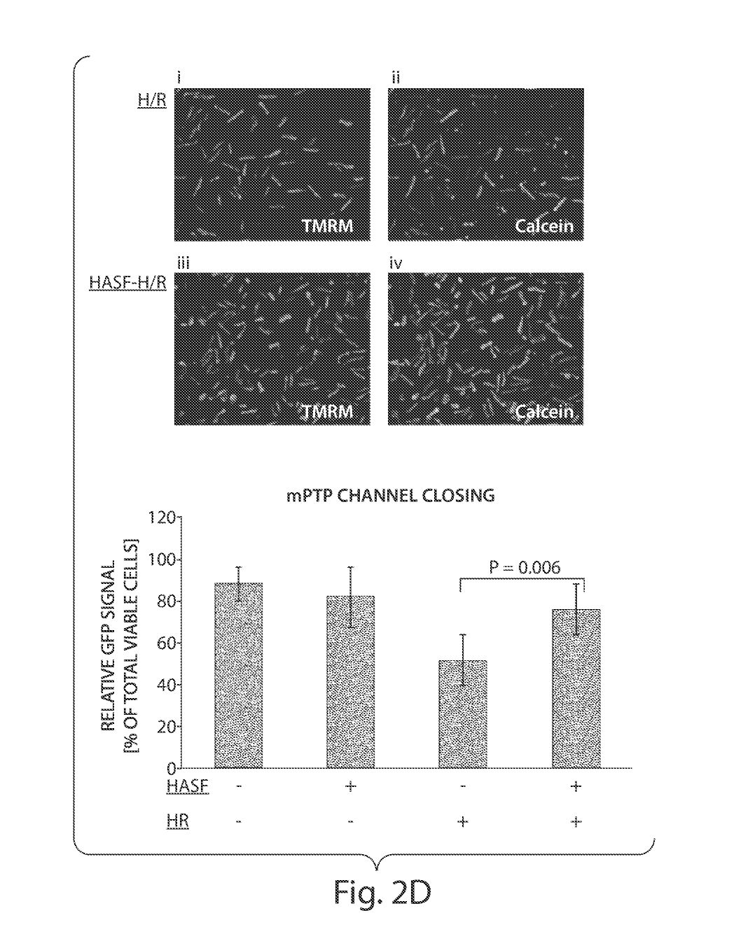

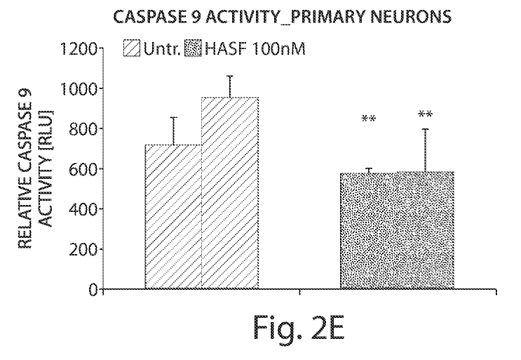

To functionally characterize HASF, human recombinant protein was produced in bacteria, purified, and tested its effect on cardiomyocyte and neuronal cells survival under conditions of stress. Initial tests in H9C2 cardiac myoblasts showed that 10 nM of HASF recombinant protein decreased significantly (˜50%) the H₂O₂ induced apoptosis as evidenced by Annexin V/PI staining, (FIG. 2A); this effect was comparable to 10 nM of human IGF recombinant protein.

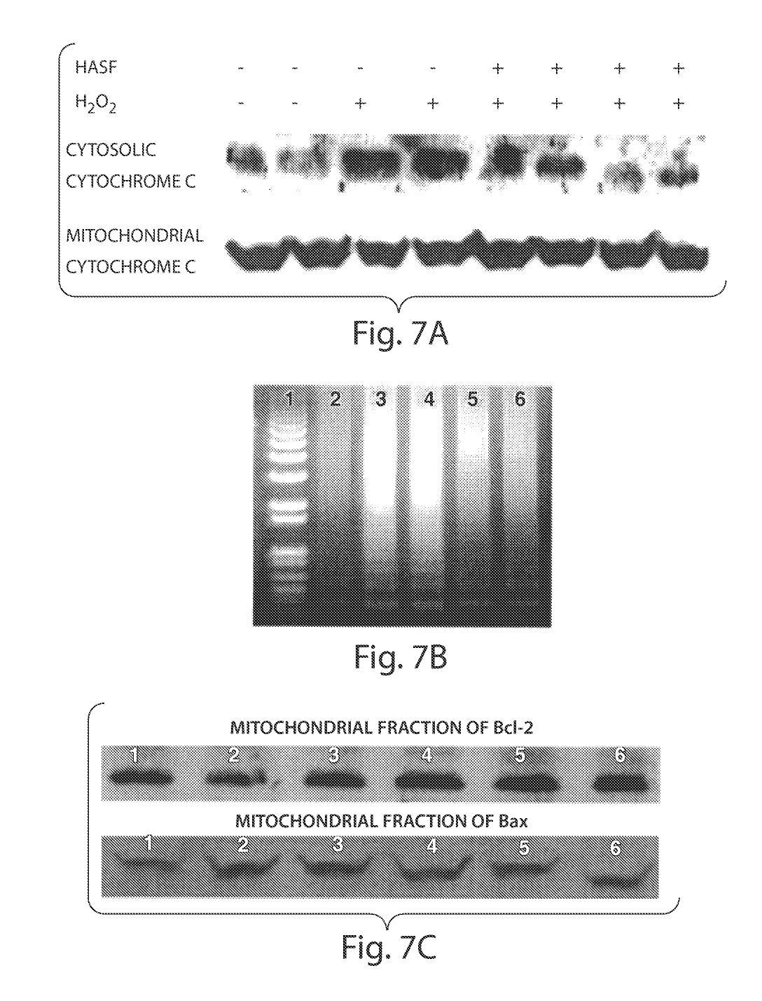

Studies were carried out to further elucidate the role of HASF in the mitochondrial apoptotic pathway using primary adult rat cardiomyocytes. In response to stress, the mitochondrial membrane is permeabilized either by opening of the mitochondrial apoptosis-induced channel MAC or opening of the permeability transition pore PTP (mPTP), resulting in the release of cell death mediators into the cytosol and activation of caspases. When adult rat cardiomyocytes were pre-incubated with 10 nM of HASF for 30 min and then subjected to treatment with 100 μM of H₂O₂, the levels of both the initiator Caspase 9 and effector Caspase 3/7 were substantially reduced compared to control samples (˜38% reduction of Caspase 9 and ˜45% reduction of Caspase 3/7 at 5 h of H₂O₂ respectively, FIGS. 2B and C). The reduction in Caspase activities was accompanied by prevention of cytochrome C release into the cytosol, increased levels of mitochondrial anti-apoptotic Bcl-2 protein, as well as reduction of DNA fragmentation (FIG. 7A-C). Furthermore, when adult rat cardiomyocytes were treated with HASF prior to hypoxia/reoxygenation in vitro, mPTP channel opening was reduced compared to control untreated samples, indicating better survival (FIG. 2D).

To examine if HASF has broad cytoprotective effects on cells other than cardiomyocytes, experiments were carried out on primary embryonic neuronal cells. Primary neurons were pretreated for 30 min with HASF followed by challenge with H₂O₂ (50 μM) for 30 min. Similar to cardiac cells, H₂O₂ exposure resulted in neuronal cell injury as documented by increased Caspase 9 levels; and pretreatment with HASF significantly attenuated Caspase 9 levels by 20-40%, P<0.05 (FIG. 2E).

### HASF Activates PKC and MAPK Pathway

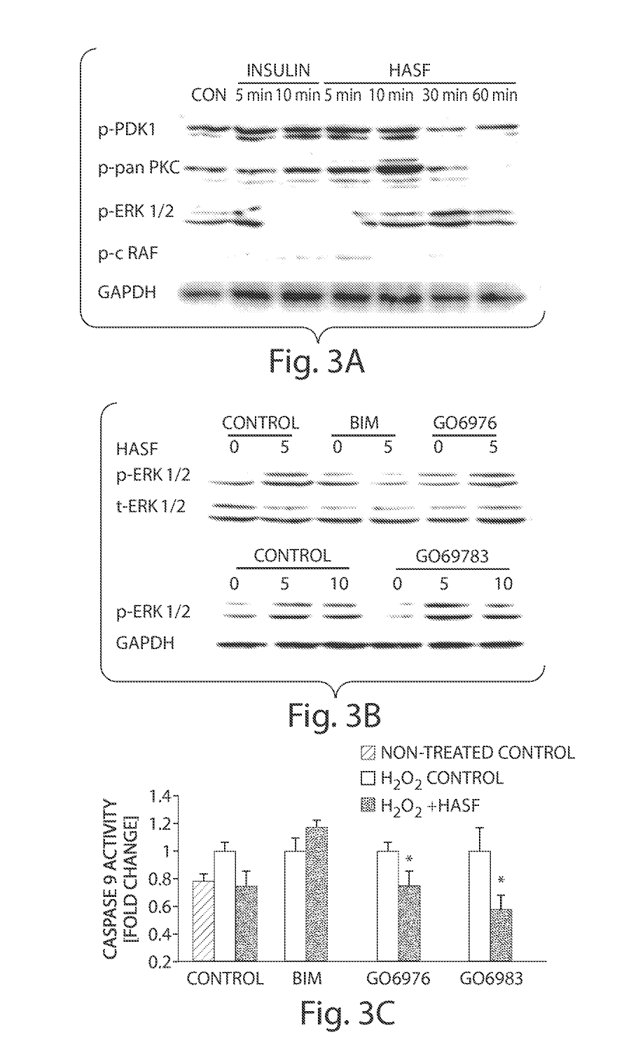

To elucidate the intracellular mechanisms underlying the anti-apoptotic effects of HASF, studies were carried out to determine the activity of several cell signaling pathways that play pivotal roles in determination of cell fate. Primary adult rat cardiomyocytes were stimulated with 100 nM of HASF for 0-60 minutes. As shown in FIG. 3A, HASF increased dramatically the phosphorylation of PKC, Raf, and ERK 1/2 within 5-10 minutes of treatment. In contrast, the phosphorylation of PDK1, a key enzyme mediating activation of Akt pathway, was not increased by HASF.

To evaluate whether PKC is essential for the HASF induced cell protective effects, adult rat cardiomyocytes were pretreated with several PKC inhibitors, including BIM, Gö6976, and Gö6983 and the effects in HASF mediated ERK activation was monitored. BIM is a nonspecific inhibitor of PKC α, β, γ, σ, ζ, and μ; Gö6976 is a nonspecific inhibitor of PKC α, β1, ζ; and Gö6983 inhibits α, β, γ, σ, and μ. The results presented in FIG. 3B reveal that BIM, but not Gö6976, or Gö6983, abolished HASF-mediated ERK1/2 phosphorylation. More importantly pre-incubation with BIM also abolished the protective effects of HASF in cardiomyocytes under stress (FIG. 3C).

### PKC Epsilon Plays an Essential Role in HASF-Mediated ERK Activation and Anti-Apoptotic Effects

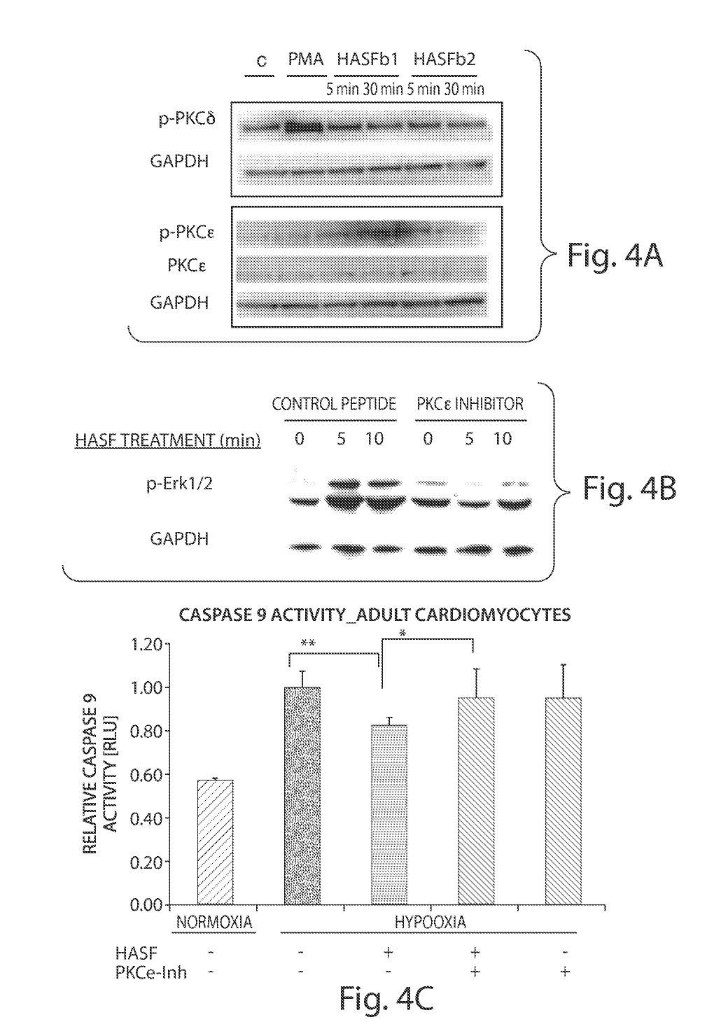

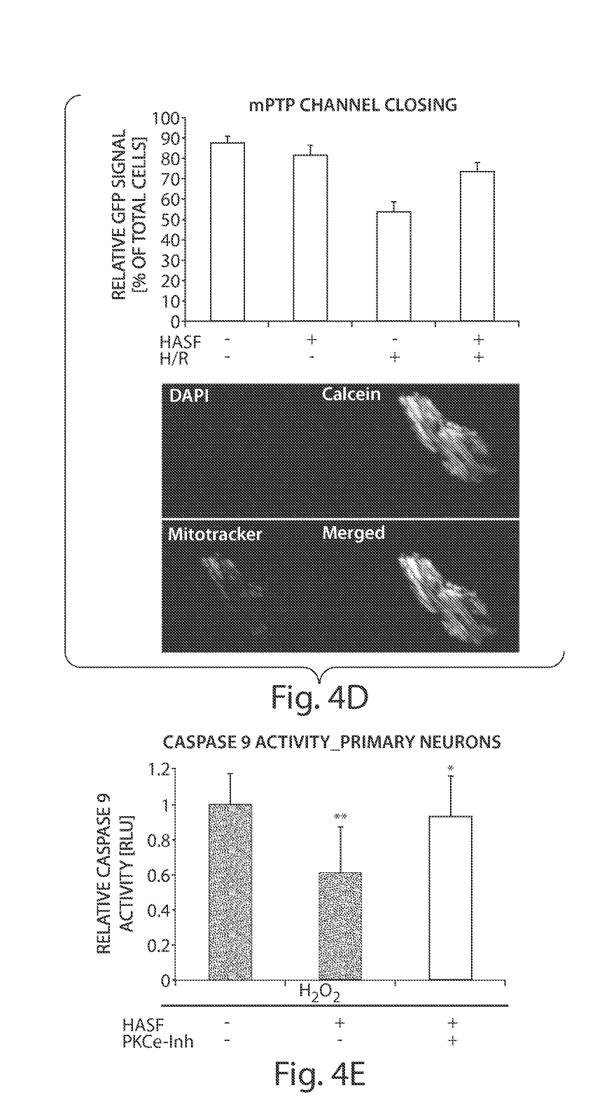

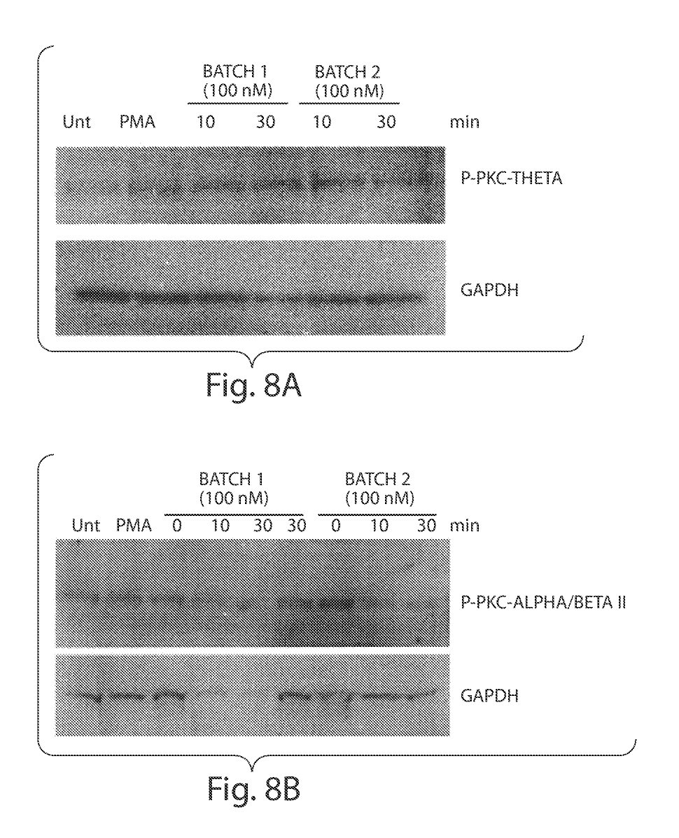

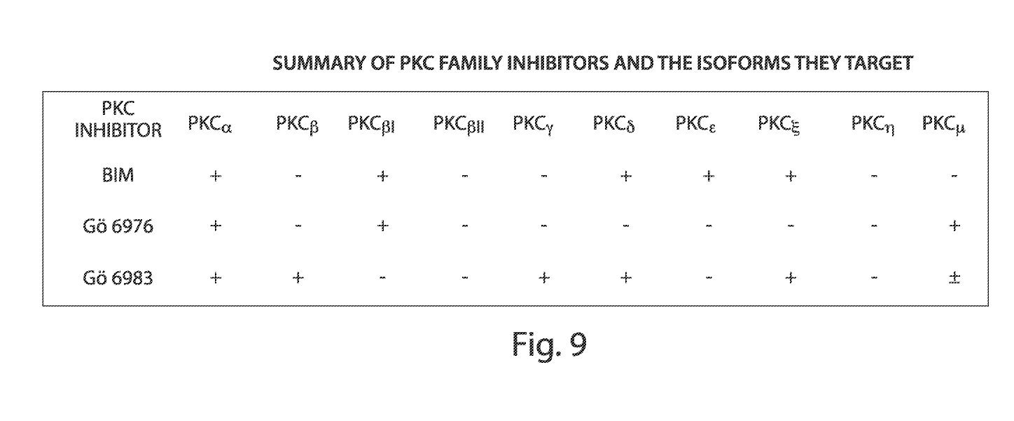

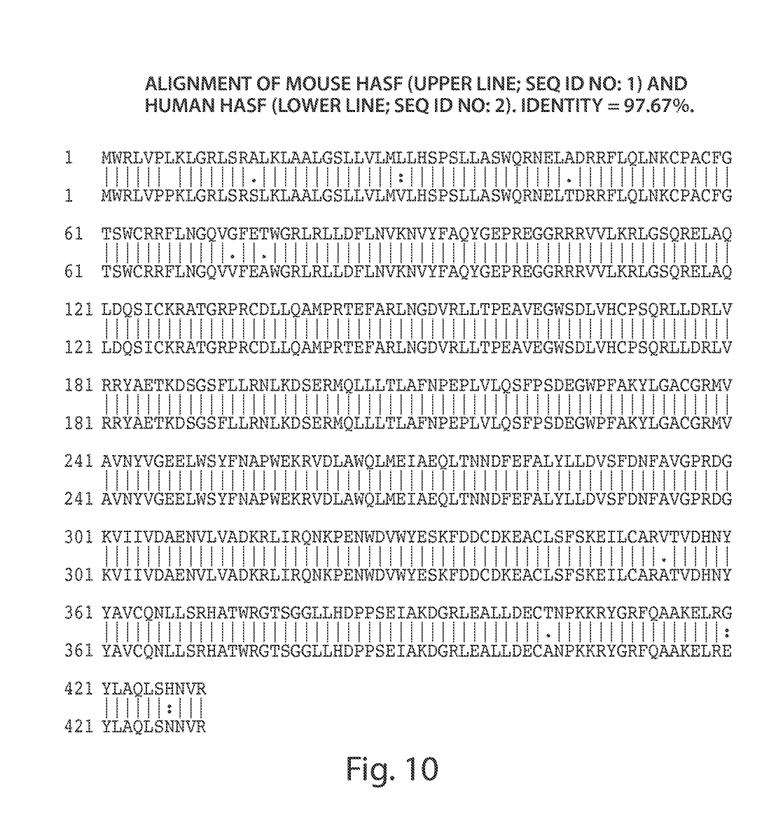

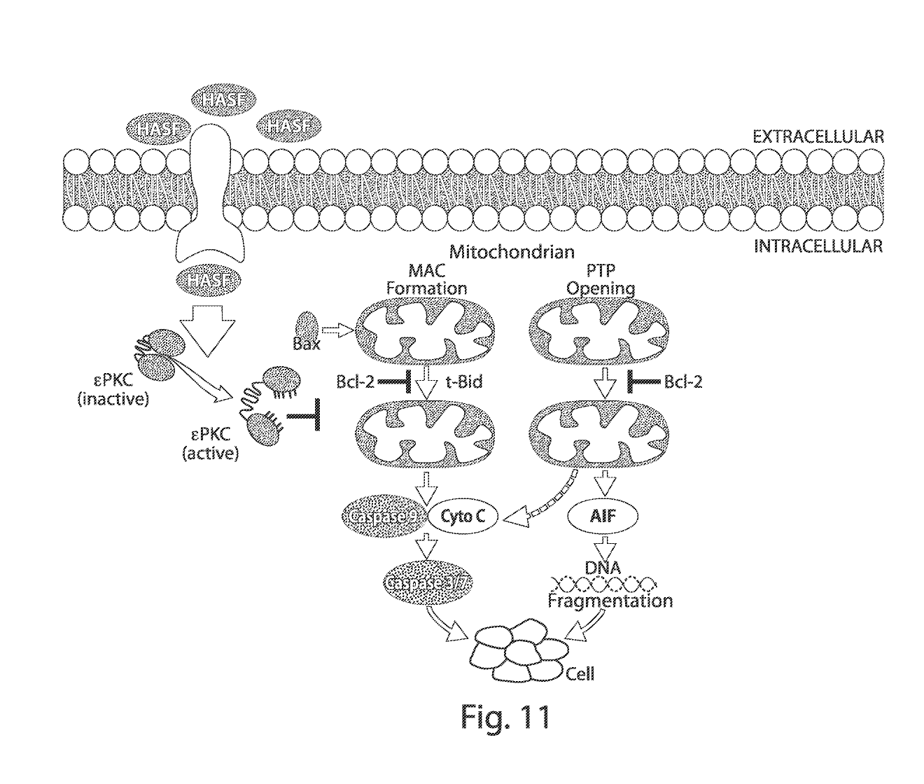

Since the PKC epsilon is the only isoform differentially affected by BIM but not the other inhibitors used (FIG. 9), studies were carried out to evaluate the role of PKCe as a pivotal factor in HASF-mediated protective effects by investigating if HASP selectively induces PKCE phosphoylation. As in the previous experiment, adult rat cardiomyocytes were stimulated with 100 nM HASF, and examined for PKC α/β, PKC σ, PKC σ/λ, PKC θ, and PKCe phosphorylation. As shown in FIG. 4A, HASF increased the phosphorylation of PKCe selectively but did not affect the activity of any of the other isoforms tested (FIGS. 8A,B). To provide further evidence for the crucial role of PKCE as mediator of HASF actions, tests were carried out to determine if selective inhibition of PKCE could block the HASF-mediated effects. Cells subject to ischemic stress (H₂O₂) and incubated 5-10 minutes with or without 100 nM HASF were pretreated with either 10 μM selective PKCe translocation inhibitor peptide or its scramble control peptide for 20-25 minutes. The effects in ERK activation were monitored. As shown in FIG. 4B, PKCe translocation inhibitor peptide, but not the control peptide treatment eliminated the HASF induced ERK1/2 phosphorylation. Moreover, preincubation of cells with the PKCE translocation inhibitor peptide also specifically prevented the cytoprotective effect of HASF on cardiomyocytes subjected to H₂O₂ oxidative stress (FIG. 4C). Similarly when adult rat cardiomyocytes underwent hypoxia/reoxygenation in vitro, preincubation of HASF treated cells with the PKCe translocation inhibitor peptide resulted in dramatic prevention of the HASF effects in preservation of mPTP closing and increased cell death compared to HASF only treated or cells treated both with control peptide and HASF (FIG. 4D).

To examine whether PKCe also mediates the observed cytoprotective effects of HASF on cells other than cardiomyocytes, the selective peptide inhibitor experiments were repeated using primary embryonic rat neurons. Primary neurons were pretreated with either 178 μM selective PKCe translocation inhibitor peptide or its scramble control peptide for 10 minutes. The cells were then treated for 30 min with HASF followed by challenge with H₂O₂ (50 μM) for 30 min. As shown in FIG. 4E, treatment with HASF significantly reduced the Caspase 9 levels in the challenged neuronal cells and these effects were blocked by the PKCE translocation inhibitor.

### HASF Decreases Apoptosis, Reduces Tissue Damage, and Fibrosis In Vivo in a Rat Model of Myocardial Infraction

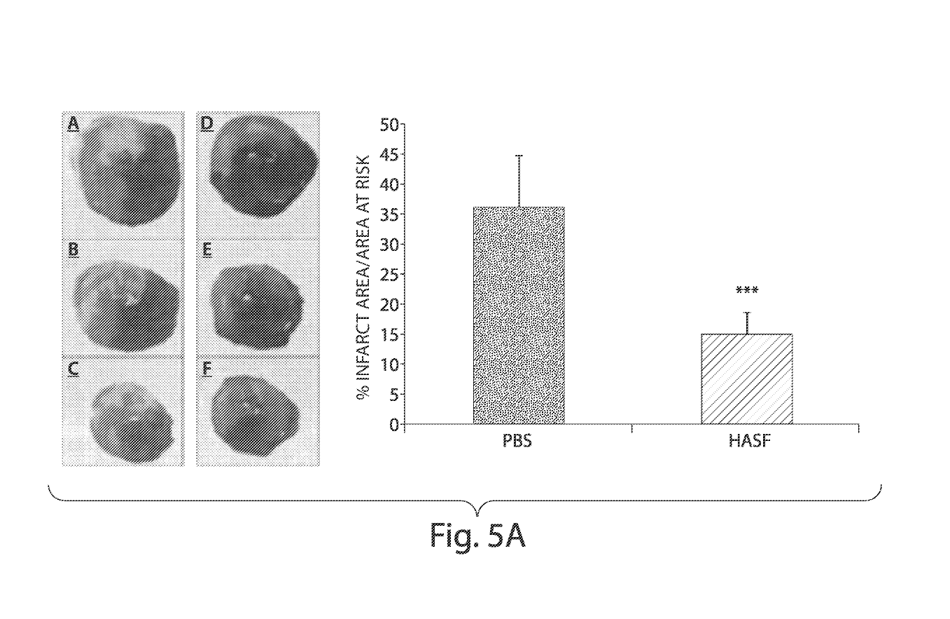

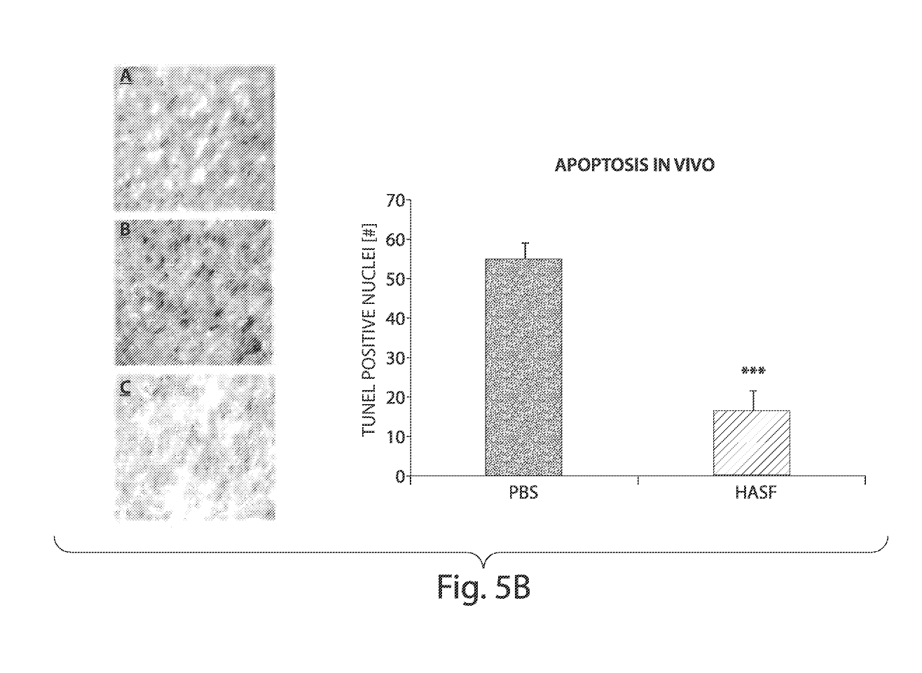

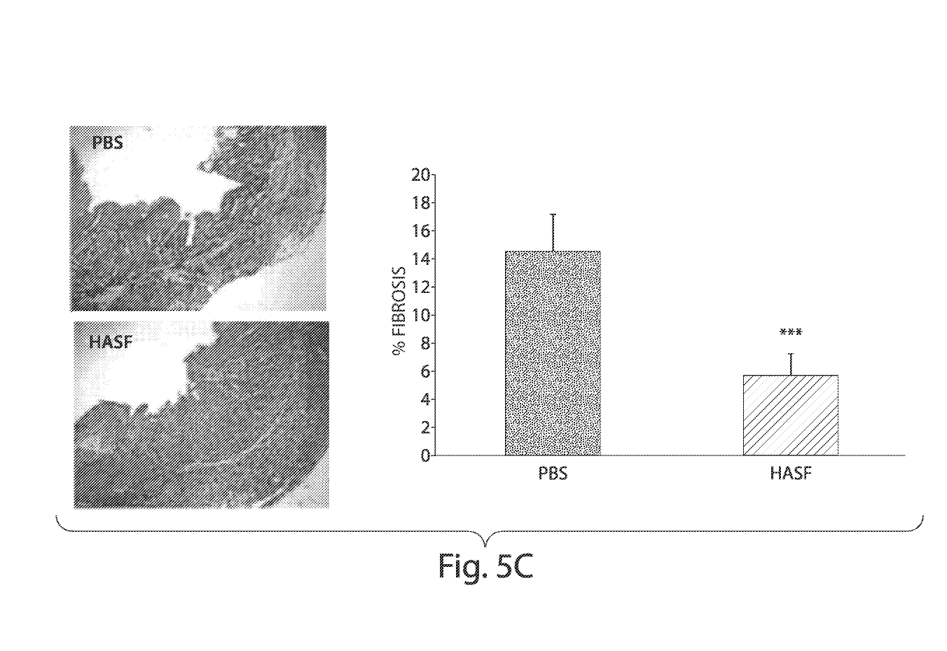

Experiments were carried out to determine whether the significant in vitro protective effects of HASF are observed in an art recognized in vivo in an animal model of tissue injury. The rat myocardial infarction is a well validated model of ischemic tissue damage. As shown in FIG. 5A, 30 min of ischemia followed by 24 h reperfusion resulted in significant myocardial infarct as evidenced by triphenyl tetrazolium chloride (TTC) and Evan's Blue stained cross sections in rat hearts. Intramyocardial injection of 1 μg of HASF recombinant protein immediately after the LAD ligation resulted in a dramatic ˜58% reduction in the infarct size. TUNEL staining in tissue sections from HASF treated animals revealed that apoptosis within the peri-infarct region was reduced significantly (˜69% reduction of the number of TUNEL positive nuclei as shown FIG. 5B).

Moreover, analysis of myocardial fibrosis using Masson's Accustain Trichrome staining of rat heart sections 4 weeks after ischemia/reperfusion showed that HASF treated hearts had a significant reduction of scar to only 5.7±1.5% of LV area translating to a 61% reduction of infarct fibrosis compared to the control untreated animals (FIG. 5C).

### HASF Induces a Protective Response Against Ischemic Tissue Damage and Promotes Cell Survival Under Conditions of Ischemia

HASF was found to protect cells from stress and hypoxia damage via specific activation of PKCe signaling mechanisms. Bioinformatic analysis of HASF sequence showed that it possesses a typical N-signal peptide without any hydrophobic transmembrane domains as seen in most classical secreted proteins. Western blotting of conditioned medium from Akt MSCs confirmed that HASF is a secreted protein. Protein sequence alignment of human and mouse HASF showed a ˜98% homology, indicating a high conservation of this protein between species during evolution.

To functionally characterize this gene and elucidate its potential role in stem cell mediated effects we cloned the human cDNA, expressed it in both bacterial and/or mammalian expression systems and purified the recombinant protein. HASF protected H9C2 myocytes in vitro against H₂O₂ induced early apoptosis as detected by Annexin V/PI staining. These data were corroborated by further experiments in adult rat cardiomyocytes and neuronal cells. The addition of recombinant protein dramatically inhibited Caspase 9 and Caspase 3/7 activities in H₂O₂ induced apoptosis and also prevented DNA fragmentation. The release of cytochrome C from mitochondria into cytosolic compartment was also greatly reduced by pre-incubation with HASF. In addition, HASF also maintained mitochondrial Bcl-2 protein level during H₂O₂ induced apoptosis but did not prevent the translocation of Bax protein from cytosol into mitochondria. Furthermore, addition of HASF protein preserved cell viability and significantly prevented mitochondrial permeability transition pore (mPTP) opening in rat cardiomyocytes subjected to hypoxia/reoxygenation injury. Intramyocardial injection of HASP into rat heart undergoing ischemia/reperfusion significantly reduced apoptosis in vivo, leading to a significant reduction of myocardial infarct size as compared with PBS injected animals. HASF treatment also resulted in much smaller myocardial scars 4 weeks later.

These data demonstrate that HASF protein is useful to promote cell survival through inhibition of programmed cell death pathways including the inhibition of caspase cascade, impairment of mitochondrial integrity, reduction of cytochrome C release, and eventually apoptosis.

The data address the mechanism underlying the protective effects of HASF in primary adult rat cardiomyocytes and embryonic neuronal cells and revealed that HASF activated PKC/ERK pathway and that its cardioprotective effects are mediated by specific activation of the PKCe isoenzyme. The PKC protein family is composed of at least eleven isozymes that have been categorized into three subfamilies based on their homology and biochemical properties. The classical PKCs (α, βI, βII, and γ) are diacylglycerol (DAG) and calcium-dependent enzymes; The novel PKCs (σ, θ, and η) require only DAG; and the atypical PKCs (ζ, λ) are not dependent on responsive either DAG or calcium, but are activated by other lipid-derived second messengers. The phosphorylation state of isozymes as well as their cellular localization determined by their interaction with their RACK partners are critical determinants of their activity and functional specificity. Members of each family can have different even opposing functions. In the context of normal cardiac development and ischemia/reperfusion injury, the PKC family has been found to play an important but complex role. Two members of the family, PKCdelta and epsilon, play opposing roles in ischemia/reperfusion injury. Activation of PKCσ during reperfusion induces cell death, whereas activation of PKCe diminishes apoptosis. Further experiments have also demonstrated that PKCe is an important mediator of preconditioning/postconditioning in the heart and brain. Using the PKCE specific antagonist, it was found that PKCe was the PKC isoform involved in mediating preconditioning-induced protection. In mice lacking PKCe, induction of preconditioning was abrogated. In brain, preconditioning significantly increased the level of hippocampal synaptosomal PKCe whose activation protected the tissue by increased synaptosomal mitochondrial respiration and phosphorylation of mitochondrial respiratory chain proteins.

Preconditioning and post conditioning refer to the observation that the application of non-lethal brief episodes of ischemia and reperfusion prior or just immediately after a sustained ischemic event confers cardiac tissue protection. Still, the clinical translation of this a phenomenon is limited due the reluctance of purposely creating an ischemic myocardium in humans and the need for high level of precision and timely intervention. A pharmacologic alternative for mimicking the effects of pre-conditioning is achieved through the use of HASF. PKCe acts through activation of prosurvival signaling pathways such as ERK, regulation of sarcKATP and connexin 43 in the cell membrane and direct or indirect activation of mitochondrial protective signals such as mitoKATP channels and mPTP (mitochondrial permeability transition pore) preservation regulation of protective mitochondrial targets such as of mPTP. In particular the inhibition of mPTP opening during the first minutes of reperfusion is a key event in pre- or post-conditioning events whereas both PKCe and ERK prosurvival pathways act at least partially through regulation of the mPTP opening. PKCe might also act through activation of ERK 1/2.

The data described herein support the use of HASF as a highly specific regulator of preconditioning in cardiomyocytes and neuronal cells. HASF is the only protein to-date that selectively activates PKCe leading to the activation of ERK pathway as well as the inhibition of mPTP thereby preserving cell viability. Specific inhibition of the PKCe, but not of the other PKC isoenzymes, attenuated the HASF mediated effects. In accordance, administration of HASF in a rat in vivo model of ischemia/reperfusion injury reduced cell death, decreased infarct size and eventually led to reduced scarring.

Specific pharmacological agents that specifically induce of pre/post conditioning are lacking. HASF represents therapeutic agent which fully mimics preconditioning protection of ischemic myocardium. The data in primary rat neuronal cells also show that the protective effects of HASF are not limited to myocardial cells. Thus, HASF has a broader therapeutic role in tissue repair and regeneration.

As in the heart, HASF is useful for the prevention of ischemic cell death and reperfusion injury of the brain and organs such as kidney, intestines and others. HASF is also useful to treat or reduce the severity of pathological conditions of tissue injury, development or degeneration, e.g., conditions such as autism.

### Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims.

Other aspects, advantages, and modifications considered to be within the scope of the following claims.

1. A method of inducing a protective response against ischemic tissue damage, comprising administering to subject a composition comprising an HASF, wherein said HASF selectively activates Protein Kinase C epsilon (PKCe).

2. The method of claim 1, wherein said subject is at risk of developing an ischemic event.

3. The method of claim 1, wherein said tissue is cardiac tissue.

4. The method of claim 1, wherein said tissue is a non-cardiac tissue.

5. The method of claim 1, wherein said tissue is neuronal tissue.

6. The method of claim 1, wherein said tissue is neuronal tissue prior to an ischemic event.

7. The method of claim 1, wherein said HASF is administered at least one year prior to an ischemic event.

8. The method of claim 1, wherein said HASF is administered at least three times prior to an ischemic event.

9. The method of claim 1, wherein said HASF is administered within 24 hours after an ischemic event.

10. The method of claim 1, wherein said non-cardiac tissue is kidney, brain, skeletal-muscle, lung, liver, or skeletal tissue.

11. The method of claim 1, wherein said HASF is administered at a dose that increases tissue activity or expression of phosphor-ERK 1/2.

12. The method of claim 1, wherein said HASF is administered at a dose that increases tissue activity or expression of protein kinase C (PKC) epsilon.

13. The method of claim 1, wherein said HASF comprises the amino acid sequence of SEQ ID NO:1 or 2 or a fragment thereof.

14. A method of inducing a protective response against ischemic tissue damage, comprising contacting said tissue with a composition comprising an HASF.
